(19) United States
(12) Patent Application Publication
(10) Pub. No.: US 2009/0270823 A1
(43) Pub. Date: Oct. 29, 2009

(54) Absorbent pads and method of making such pads

(75) Inventor: Joseph Meir Meizelman, Zug (CH)

Correspondence Address: MARTIN D. MOYNIHAN d/b/a PRTSI, INC., P.O. BOX 16446, ARLINGTON, VA, 22215, US

(73) Assignee: Bright Technologies Corp. Inc., Zug (CH)

(21) Appl. No.: 12/149,159

(22) Filed: Apr. 28, 2008

(51) Int. Cl. A61F 13/84; C09J 5/02

(52) U.S. Cl. 604/359; 156/306.3

(57) ABSTRACT

A method of making an absorbent pad, and the absorbent pad so made, by bonding together a lower layer, an intermediate layer of an absorbent pulverized pulp, and an upper layer; characterized in that the intermediate layer also includes a perfumed deodorant powder mixture mixed with the absorbent pulverized pulp to reduce odors and to prevent skin erythema during the use of the absorbent pad by a person.

### FIELD AND BACKGROUND OF THE INVENTION

The present invention relates to methods of making absorbent pads, and also to absorbent pads made by such methods.

Absorbent pads, sometimes called underpads, are used in a wide variety of applications, including: medical applications, for wound care and hospital underpads; feminine hygiene, such as sanitary napkins, panty shields; baby and adult diapers; food packaging, such as soak pads for meat and fish packaging; industrial filtration, such as for kerosene, hydraulic oils, etc. Many methods have been developed for making such absorbent pads as described, for example, in U.S. Pat. Nos. 3,692,622, 6,675,702, and 6,675,703. However, efforts are continuously being made to improve the properties of such absorbent pads, as well as their method of manufacture.

### OBJECT AND BRIEF SUMMARY OF THE PRESENT INVENTION

An object of the present invention is to provide a method of making an absorbent pad having improved properties over the absorbent pads that have heretofore been developed. Another object of the invention is to provide a method of making such absorbent pads.

According to a broad aspect of the present invention, there is provided a method of making an absorbent pad comprising bonding together a lower layer, an intermediate layer of an absorbent pulverized pulp, and an upper layer; characterized in that the intermediate layer also includes a deodorant powder mixed with the absorbent pulverized pulp to reduce odors produced during the use of the absorbent pad by a person.

According to a further feature in the described preferred embodiments, the deodorant mixture powder includes a deodorant agent to reduce odors, and an anti-inflammatory agent to prevent skin erythema during the use of the absorbent pad by a person.

In the described preferred embodiments, the deodorant agent is talc powder and the anti-inflammatory agent is zinc oxide or aloe vera extract. Preferably, the deodorant powder mixture is present from 5 to 45 per cent by weight of the intermediate layer. Also in the described preferred embodiments, the deodorant agent is present from about 60 to 80 per cent by weight of the deodorant mixture powder. The deodorant mixture powder may also include a perfume fragrance, e.g. from 1 to 3 per cent by weight of the powder mixture, to mask perspiration and/or urine odors.

According to further features in the described preferred embodiments, the intermediate layer further includes particles of a super absorbent polymer, for example a cross-linked sodium polyacrylate, preferably applied between sub-layers of the pulverized pulp.

According to further features in the described preferred embodiments, the layers are bonded together by pressure applied by embossing rollers. In addition, the intermediate layer has an initial water content of over 5 per cent, preferably between 7-10 per cent, before the layers are bonded together.

According to further features in the described preferred embodiments, the upper and lower porous layers is an unwoven tissue layer or a porous plastic film, such as micro-perforated polyethylene film.

Another embodiment is described wherein the three-layer pad defines a core in which the lower porous layer is backed by a backing layer of a non-porous plastic film, such as polyethylene. In the latter embodiment, the absorbent pad also includes an overlying porous layer over the upper porous layer and pressure bonded to the backing layer along margins extending along the four sides of the core. The overlying porous layer may also be an unwoven tissue layer or a porous plastic film.

According to another aspect of the present invention, there is provided an absorbent pad comprising a lower layer, an intermediate layer of an absorbent pulverized pulp, and an upper porous layer all bonded together by pressure; characterized in that the intermediate layer also includes a deodorant mixture powder mixed with the absorbent pulverized pulp to reduce odors and to prevent skin erythema produced during the use of the absorbent pad by a person.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a block diagram schematically illustrating a production line for one method of making absorbent pads in accordance with the present invention;

FIG. 2 is a sectional view illustrating the absorbent pad made according to the method of FIG. 1;

FIG. 3 illustrates the embossing rollers of the block diagram of FIG. 1;

FIG. 4 is a side view illustrating the surface of one of the embossing rollers of FIG. 3;

FIG. 5 is an enlarged fragmentary view more particularly illustrating the surface of the embossing rollers;

FIG. 6 is a block diagram schematically illustrating another production line for another method of making absorbent pads in accordance with the present invention; and

FIG. 7 is a sectional view illustrating the absorbent pad made in accordance with the method of FIG. 6.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

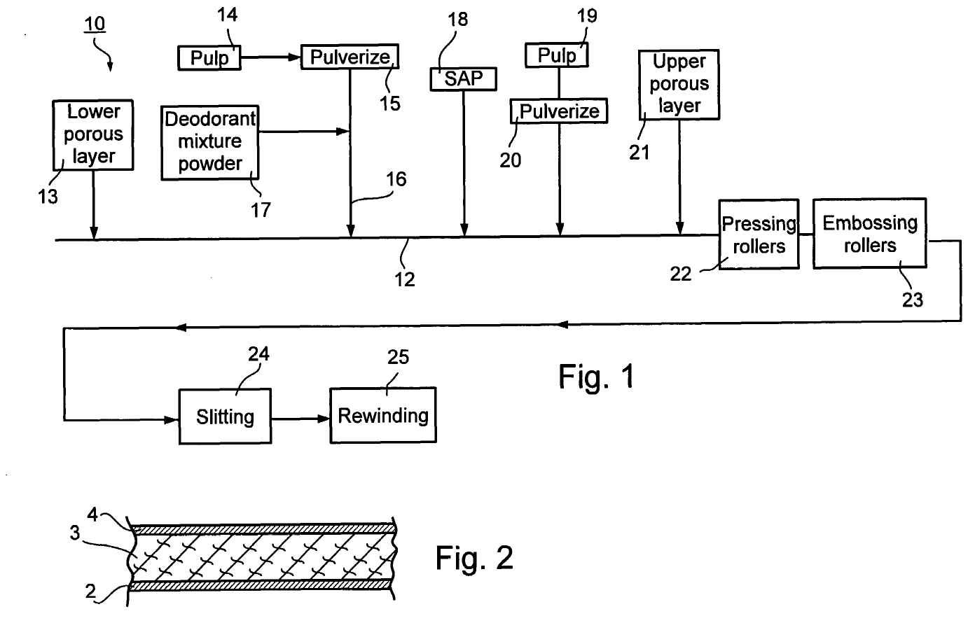

### The Embodiment of FIGS. 1 -5

FIG. 1 schematically illustrates a production line, generally designated 10, for making an absorbent pad in accordance with the present invention, and the absorbent pad made according to FIG. 1 is illustrated in FIG. 2.

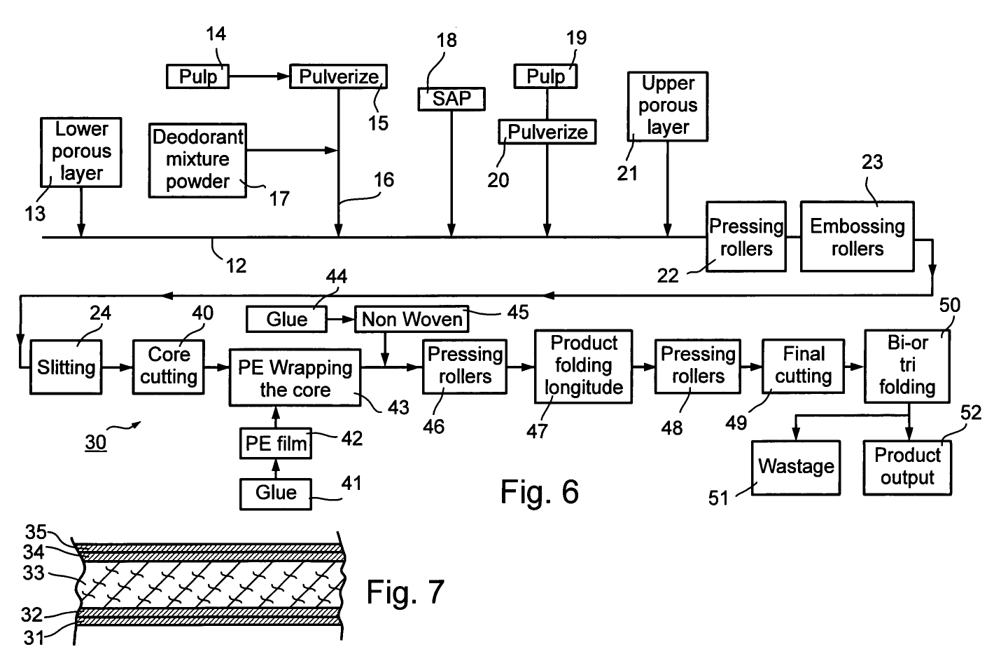

The absorbent pad produced by the production line 10 of FIG. 1 and illustrated in FIG. 2 is a three-layer pad, including a lower porous layer 2; an intermediate layer 3 of an absorbent pulverized pulp; and an upper porous layer 4. As will be described more particularly below, the three-layer absorbent pad of FIGS. 1 and 2 may be used alone, or it may be used as a core for producing an absorbent pad of more than three layers, such as the five-layer absorbent pad of FIGS. 6 and 7.

Thus, the production line 10 shown in FIG. 1 includes a web 12 moved through a number of stations at which various materials are applied thereto and processed thereon to produce the three-layer pad of FIG. 2.

The first station is shown at 15, whereat a porous layer is deposited on web 12 to form the lower porous layer 2 in the three-layer pad of FIG. 2. This lower porous layer 2 may be an unwoven tissue layer, or a porous plastic film such as polyethylene formed with micro-perforations.

Next, pulp 14 is pulverized at 15 wherein it is crushed. The pulp is separated into cellulose fibers and applied via a vacuum conveyor 16 onto the porous layer 2. While the fibers move through the vacuum conveyor, a quantity of a deodorant mixture powder 17 is added to the fibers so as to be mixed therewith when deposited on the porous layer 2.

The deodorant mixture powder 17 includes a deodorant agent, and anti-inflammatory agent, and preferably also a perfume fragrance. The deodorant agent is preferably a talc powder, but may also be another deodorant material such as triclosan. The anti-inflammatory agent is included to prevent skin erythema. A preferred material is zinc oxide, but can also be another anti-inflammatory agent, such as urea, or allantoin or aloe vera extract. The perfume fragrance is included to mask perspiration, urine, or other odors when the absorbent pad is used by a person.

The deodorant mixture powder 17 may generally be present from 5 to 45 per cent, preferably about 20 per cent, of the intermediate layer 3. The deodorant agent is generally present from 60 to 80 per cent, preferably about 75 per cent, by weight, of the deodorant mixture powder; the anti-inflammatory agent may be present from 20-30%, preferably about 23%, of the deodorant mixture powder; and the perfume fragrance is present, by about 1 to 5 per cent by weight, preferably about 2 per cent, of the deodorant mixture powder.

Following the deposition of the pulverized pulp 15 mixed with the deodorant mixture powder 17, a super absorbent polymer (SAP) material 18 is deposited (optionally with odor control). Such polymers are well known and have the capability of absorbing and retaining extremely large amounts of a liquid relative to their own mass. The water absorbing polymers, classified as hydrogels, absorb aqueous solutions through hydrogen bonding with the water molecule. Preferably, the SAP material is a cross-linked sodium polyacrylate made from the pulverization of acrylic acid blended with sodium hydroxide in the presence of an initiator. It will be appreciated, however, that other materials may be used to make the super absorbent polymer, such as polyacrylamide copolymer, starch grafted copolymer of polyacrylonitrile, etc.

Following the deposition of the SAP material 18, another quantity of pulp 19 pulverized at 20 is applied, and then the upper porous layer 4 is applied at station 21. Preferably, the upper porous layer 4 is of the same material as the lower porous layer 2.

After the foregoing layers have been applied, the so-formed pad is passed through pressure rollers 22, embossing rollers 23, and slitting rollers 24, which slits the edges of the pads, before being wound on a roll as shown at 25.

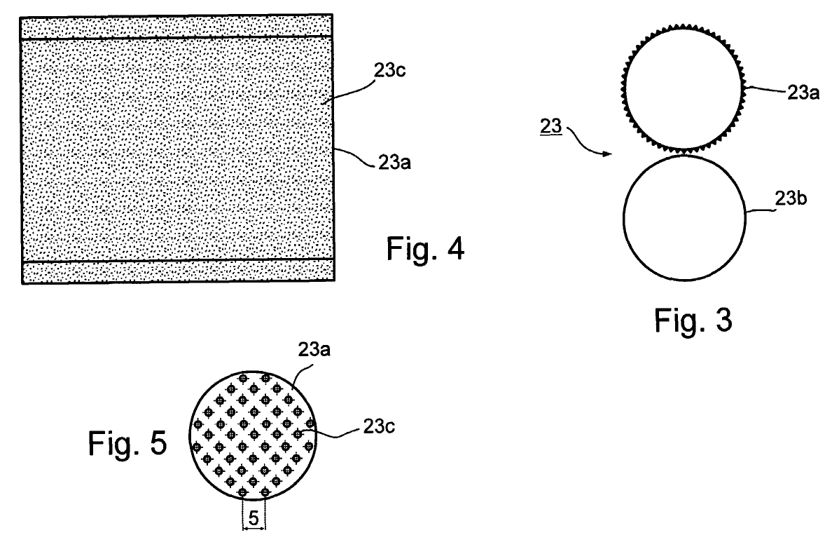

Pressure rollers 22 may be a pair of calendar rollers that bond the various layers together by pressure. Embossing rollers 23 may be a second pair of calendar rollers with embossing patterns on them to emboss the web as desired. FIGS. 3-5 illustrate one construction of embossing rollers, therein designated 23a, 23b, that may be used. As shown particularly in FIGS. 4 and 5, such embossing rollers include semi-cylindrical projections, shown at 23c (FIG. 5) projecting from the outer surfaces of both rollers to produce semi-spherical embossments in the three-layer pad shown in FIG. 2. Rollers 22 and 23 may be heated to enhance the bonding and embossing operations.

As indicated above, the absorbent pad illustrated in FIG. 2 may be used as a separate absorbent pad for certain applications, in which the pulverized pulp and SAP material in the intermediate layer 3 effectively absorb large quantities of liquids, while the deodorant powder mixture 17, added to the pulverized pulp of the intermediate layer 3, effectively prevent skin erythema and reduce odors produced during the use of the absorbent pad by a person, while the perfume fragrance masks perspiration or other (e.g. urine) odors.

### The Embodiments of FIGS. 6 and 7

FIG. 6 illustrates a production line, generally designated 30, for a method of making an absorbent pad of a larger number of layers, in which the absorbent pad of FIG. 2 constitutes the core; and FIG. 7 illustrates an absorbent pad constructed according to FIG. 6. The core of the FIG. 7 pad may be made off-line according to the block diagram of FIG. 1; alternatively, it may be part of an inline production method as illustrated in FIG. 6.

Thus, as shown in FIG. 7, the absorbent produced by the production line of FIG. 6 is a five-layer pad, rather than a three-layer pad as shown in FIG. 2. The five-layer pad of FIG. 7 includes a backing layer 31 of a non-porous plastic film, such as polyethylene; a lower porous layer 32 of the material of porous layer 2 in FIG. 3; an intermediate layer 33 including the paper pulp and super absorbent polymer (SAP), and also the deodorant powder mixture, as intermediate layer 3 in FIG. 2; an upper porous layer 34 of the same material as porous layer 4 in FIG. 2; and a top layer 35 of non woven porous layer material like spunbond or thermobond.

The first part of the inline production process illustrated in FIG. 6 is substantially the same as station 13 in FIG. 1, and similarly applies the lower porous layer 32 in FIG. 7. The intermediate layer 33 and the upper layer 34 correspond to layers 3 and 4 in the three-layer pad illustrated in FIG. 2.

The absorbent pad illustrated in FIG. 4 includes two additional layers, namely backing layer 31, underlying the lower porous layer 32, and constituted of a non-porous plastic film, such as of polyethylene; and an upper porous layer 35 overlying porous layer 34. The upper porous layer 35 may also be made of the same material as porous layers 32 and 34.

The first part of the production line in FIG. 6, up to the slitting operation at 24, is substantially the same as described above with respect to FIG. 1 and therefore corresponding parts are correspondingly numbered. During this first part of the production line, a three-layer absorbent pad is produced, constituted of layers 32, 33 and 34 in FIG. 7, corresponding to the three-layer absorbent pad of FIG. 2.

Whereas the slitting operation 24 in FIG. 1 merely trims the edges of the pad, in the core cutting operation of FIG. 6, the produced pad is cut into the desired length. A layer of glue 41 is then applied to the non-porous plastic film 42, such as of polyethylene, and the latter film is applied as layer 31 to the backing layer 32, as shown in FIG. 7.

A layer of glue 44 is then applied to the upper surface of the upper porous layer 34, and the top porous layer 35 is then applied at 45 over layer 34.

Backing layer 31 and the top porous layer 35 are preferably of larger dimensions than the core constituted of layers 32, 33 and 34, so that the core is circumscribed on all four sides by borders consisting only of layers 31 and 35 bonded together.

After the absorbent pad has been so produced, it is passed between a pair of pressing rollers 46 for compacting them, then folded as shown at 47, pressed as shown at 48, and finally cut as shown at 49. The so-prepared pad may then be bi-folded or tri-folded, as shown at 50. The wastage is rejected, as shown at 51, before the pad appears as a final product at 52.

The pulp used is preferably a commercially available pulp having a humidity larger than 5 per cent, preferably 7-10 per cent. The pressure rollers are not heated so that the bonding is affected by pressure alone. Preferably, the embossing rollers shown in FIGS. 3-5, which may be heated, are used for the embossing operation.

While the invention has been described with respect to two preferred embodiments, it will be appreciated that these are set forth merely for purposes of example, and that many other variations, modifications and applications of the invention may be made.

What is claimed is:

1. A method of making an absorbent pad comprising bonding together a lower layer, an intermediate layer of an absorbent pulverized pulp, and an upper layer; characterized in that said intermediate layer also includes a deodorant mixture powder mixed with said absorbent pulverized pulp to reduce odors produced during the use of said absorbent pad by a person.

2. The method according to claim 1, wherein said deodorant mixture powder includes a deodorant agent to reduce odors, and an anti-inflammatory agent to prevent skin erythema during the use of the absorbent pad by a person.

3. The method according to claim 2, wherein said deodorant agent is talc powder, and said anti-inflammatory agent is zinc oxide.

4. The method according to claim 2, wherein said deodorant mixture powder is present from 5 to 45 per cent by weight of said intermediate layer.

5. The method according to claim 2, wherein said deodorant agent is present from 60 to 80 per cent by weight of said deodorant mixture powder.

6. The method according to claim 1, wherein said deodorant mixture powder also includes a perfume fragrance to mask perspiration and/or urine odors.

7. The method according to claim 1, wherein said intermediate layer further includes particles of a super absorbent polymer.

8. The method according to claim 7, wherein said super absorbent polymer is applied between two deposits of said absorbent pulverized pulp.

9. The method according to claim 1, wherein said layers are bonded together by pressing rollers and then by embossing rollers.

10. The method according to claim 1, wherein said intermediate layer has an initial water content of 7 to 10 percent before the layers are bonded together.

11. The method according to claim 1, wherein said upper porous layer is an unwoven tissue layer or a porous plastic film.

12. The method according to claim 1, wherein said lower porous layer is an unwoven tissue layer or a porous plastic film.

13. The method according to claim 12, wherein said porous plastic film is polyethylene.

14. The method according to claim 1, wherein said lower layer is a porous layer, and said absorbent pad includes a backing layer of a non-porous plastic film underlying said lower layer.

15. The method according to claim 14, wherein said absorbent pad includes an overlying porous layer over said upper layer and pressure-bonded to said backing layer.

16. The method according to claim 15, wherein said overlying porous layer is an unwoven tissue layer or a porous plastic film.

17. An absorbent pad comprising a lower layer, an intermediate layer of an absorbent pulverized pulp, and an upper porous layer all bonded together by pressure; characterized in that said intermediate layer also includes a deodorant mixture powder mixed with said absorbent pulverized pulp to reduce odors produced during the use of said absorbent pad by a person.

18. The absorbent pad according to claim 17, wherein said deodorant mixture powder includes a deodorant agent to reduce odors, and an anti-inflammatory agent to prevent skin erythema during the use of the absorbent pad by a person.

19. The absorbent pad according to claim 18, wherein said deodorant agent is talc powder, and said anti-inflammatory agent is zinc oxide.

20. The absorbent pad according to claim 19, wherein said deodorant powder mixture is present from 5 to 45 per cent by weight of said intermediate layer.

21. The absorbent pad according to claim 17, wherein said deodorant agent is present from 10 to 80 per cent by weight of said deodorant powder mixture.

22. The absorbent pad according to claim 17, wherein said deodorant powder mixture also includes a perfume fragrance to mask perspiration and/or urine odors.

23. The absorbent pad according to claim 17, wherein said intermediate layer further includes particles of a super absorbent polymer.

24. The absorbent pad according to claim 23, wherein said super absorbent polymer is a cross-linked sodium polyacrylate.

25. The absorbent pad according to claim 17, wherein said upper porous layer is an unwoven tissue layer or a porous plastic film.

26. The absorbent pad according to claim 17, wherein said lower porous layer is an unwoven tissue layer, or a porous plastic film.

27. The absorbent pad according to claim 26, wherein said porous plastic film is polyethylene.

28. The absorbent pad according to claim 17, wherein said lower layer is a porous layer, and said absorbent pad includes a backing layer of a non-porous plastic film underlying said lower layer.

29. The absorbent pad according to claim 28, wherein said absorbent pad includes an overlying porous layer over said upper layer and pressure-bonded to said backing layer.

30. The absorbent pad according to claim 29, wherein said overlying porous layer is an unwoven tissue layer or porous plastic film.
